(19) United States
(12) Patent Application Publication
(10) Pub. No.: US 2024/0359002 A1
(43) Pub. Date: Oct. 31, 2024

(54) PERICARDIAL ACCESS AND ANCHOR SYSTEMS

(71) Applicant: PercAssist, Inc., Santa Clara, CA (US)

(72) Inventors: Albert K. Chin, Mountain View, CA (US); Gerardo V. Noriega, Santa Clara, CA (US); Steven K. Kele, San Jose, CA (US)

(73) Assignee: PercAssist, Inc., Santa Clara, CA (US)

(21) Appl. No.: 18/439,610

(22) Filed: Feb. 12, 2024

Related U.S. Application Data
(63) Continuation of application No. PCT/US22/75100, filed on Aug. 17, 2022.
(60) Provisional application No. 63/234,964, filed on Aug. 19, 2021.
(60) Provisional application No. 63/234,982, filed on Aug. 19, 2021.
(60) Provisional application No. 63/241,599, filed on Sep. 8, 2021.

(51) Int. Cl. A61M 60/865; A61M 60/191; A61M 60/295; A61M 60/468; A61M 60/497; A61M 60/841; A61M 60/861

(52) U.S. Cl. CPC A61M 60/865; A61M 60/191; A61M 60/295; A61M 60/468; A61M 60/497; A61M 60/841; A61M 60/861; A61M 2205/04; A61M 2205/073; A61M 2210/122

(57) ABSTRACT

A pericardial access system includes an introducer sheath and a mandrel. The introducer sheath has a proximal end, a distal end, and a lumen extending therethrough. The mandrel has a proximal end, a distal end, end a handle at the proximal end. A wire electrode is disposed at the distal end of the mandrel, and a power supply is coupled to the wire electrode and configured to apply a short burst of energy sufficient to allow the wire electrode to penetrate a pericardial sac of a patient. The pericardial access system is used to deliver an implantable cardiac assist catheter connectable to an external drive unit. An anchor catheter is percutaneously advanced into a patient's pericardial sac and includes an anchor at its distal which may be expanded within the pericardial sac.

### CROSS-REFERENCE TO RELATED APPLICATIONS

This application is a continuation of PCT Application No. PCT/US22/75100 (Attorney Docket No. 56027-704.601), filed Aug. 17, 2022, which claims the benefit of U.S. Provisional Patent Application No. 63/234,982 (Attorney Docket No. 56027-704.101), filed Aug. 19, 2021; of U.S. Provisional Patent Application No. 63/234,964 (Attorney Docket No. 56027-705.101), filed Aug. 19, 2021; and of U.S. Provisional Patent Application No. 63/241,599 (Attorney Docket No. 56027-705.102), filed Sep. 8, 2021, the full disclosures of which are incorporated herein by reference.

### BACKGROUND OF THE INVENTION

1. Field of the Invention. The present invention relates generally to medical devices, systems, and methods, and more particularly, to devices, systems, and and methods for entering, exiting, and stabilizing an interventional or therapeutic device inside a patient's pericardial sac.

2. Background of the Invention. Entry through the pericardial sac (pericardium) and into the pericardial cavity is required for a variety of interventional and therapeutic cardiac procedures. Non-surgical or “minimally invasive” entry through the pericardium and into the cavity is generally performed via advancement of a hypodermic needle through the pericardial sac under fluoroscopic guidance. Needle entry is typically conducted through the subxiphoid region of the patient's abdomen where the needle is advanced across the patient's diaphragm and through the pericardium, usually at the border of the right ventricle of the heart.

The pericardium is a thin, tough fibrous membrane circumferentially enclosing the heart. Controlled pericardial entry is difficult due to the resistance encountered by the sharp needle tip as it punctures through the fibrous pericardium. Once past the pericardium, it is extremely difficult to stop needle tip advancement into the myocardium, particularly if limited intra-pericardial fluid is present in the cavity to create space between the epicardial surface and the pericardium. In one clinical study, inadvertent myocardial puncture was observed in up to 17% of percutaneous subxiphoid pericardial access procedures. In another study of 404 patients, open heart surgery was required in six patients to repair cardiac injuries sustained during pericardial needle entry.

Pericardial entry may be required for medical and surgical procedures performed on the epicardial surface of the heart. These include epicardial mapping and ablation procedures used to treat heart arrhythmias, left atrial appendage exclusion procedures performed to prevent stroke in patients with atrial fibrillation, transmyocardial revascularization procedures, injection of stem cells or gene therapy for myocardial regeneration and the like. Of particular interest to the present application, ventricular assist balloon cannulas may be introduced through the pericardium into the pericardial cavity to treat patients with congestive heart failure, as described in WO2020/176670.

WO2020/176670, commonly assigned with the present application, describes a “ventricular assist” balloon cannula configured to be inserted inside a patient's pericardial sac and positioned anterior to the patient's left ventricle. Inflation of the balloon during cardiac systole and deflation of the balloon during cardiac diastole may be conducted to increase cardiac output in patients with congestive heart failure and other conditions.

The ventricular assist balloon cannula is typically inserted through the pericardium at the inferior aspect of the heart near the apex, typically via a percutaneous subxiphoid incision or needle puncture. A distal end of the ventricular assist balloon cannula is advanced to the left lateral aspect of the heart immediately inferior to the left atrial appendage to position the balloon anterior to the left ventricle.

A fluid tight reservoir is then attached to the proximal end of the ventricular assist balloon cannula, and the reservoir implanted subcutaneously in the subxiphoid region. The balloon may be inflated via an external air pump using a large bore needle penetrated through the patient's skin and into the subcutaneous reservoir.

Such ventricular assist procedures require both successful percutaneous access to the patient's pericardial sac and stabilization of the ventricular assist balloon cannula after the cannula has been successfully introduced. It would therefore be desirable to provide improved apparatus, system, and methods for both accessing a patient's pericardial cavity for placement of a ventricular assist balloon cannula and stabilization of the ventricular assist balloon cannula after it has been properly placed. It would be further desirable if such apparatus, system, and method would further find use in other procedures requiring access and penetration into a patient's pericardial cavity and subsequent placement and stabilization of a variety of different in-dwelling device introduced via the penetration. At least some of these objectives will be met by the inventions described below.

### SUMMARY OF THE INVENTION

A device is provided to allow safe entry and exit through the pericardium under either fluoroscopic control or endoscopic visual control. The device consists of a vascular introducer sheath with an inner mandrel that extends distal to the distal end of the sheath. The portion of the mandrel that extends distal to the vascular sheath contains a short section that tapers down to a high-resistance wire arc that glows red hot when actuated by a battery operated power source in the handle on the proximal end of the mandrel. For pericardial entry and exit, the wire arc is pressed against the pericardium and the control switch activated to cause the wire arc to heat to a high temperature, thereby incising through the pericardium. Passage through the pericardium is appreciated by the sudden release of resistance as the hot wire punctures the fibrous pericardial membrane, and wire heating actuation is immediately discontinued by the user. The rounded contour of the wire arc prevents injury to tissue opposite the entry site of the pericardium. In contrast, the sharp tip of a hypodermic needle may readily puncture or lacerate tissue upon pericardial entry or exit. The curved contour of the hot wire and its rapid cooling rate prevent injury to the heart during sheath entry into the pericardial sac. Likewise, injury to the lung is prevented upon exit of the sheath from the pericardial sac into the pleural cavity. The height of the wire arc is also limited to a few millimeters, and initial pericardial entry is limited to this small distance. Following wire arc entry, controlled mechanical advancement of the introducer sheath and its supporting inner mandrel is performed. The tapered distal ends of the introducer sheath and the mandrel aid in sheath advancement, and a rotational motion may be performed during sheath placement.

In an alternate embodiment, hot wire actuation may be controlled by a timer circuit that cuts power to the high resistance wire after a specified duration of 1-2 seconds even upon continued depression of the power switch. With the timed heating circuit, the wire arc is pressed against the pericardium and the power switch depressed. If pericardial entry is not achieved with a single cycle of wire heating, the device position is maintained while additional heating cycles are performed to traverse the full thickness of the pericardium.

The body of the hot wire mandrel is formed of rigid inelastic polymer tubing such as polyether ether ketone (PEEK), polycarbonate, Nylon 12 or similar material. Two insulated wire electrodes extend from the handle on the proximal end of the mandrel to the distal end of the tube. The electrodes are ideally 21 gauge (0.0285″ diameter) solid copper, covered with a polymer insulation of polyvinyl chloride (PVC), polyolefin or polyethylene. A 3-5 mm long distal length of wire electrode is bare and uninsulated, and the two limbs of a high resistance Nichrome wire arc are placed in electrically conductive contact with the bare electrode ends and fixed in position via crimped outer sleeves of brass or stainless steel tubing. The Nichrome wire diameter is approximately 0.010″, and the outer diameter of its formed arc is approximately 0.11″. The crimped tubes are covered by heat shrink tubing of polyethylene terephthalate, polyolefin or other polymer, to electrically insulate both limbs leading to the Nichrome wire arc. The crimped tubes are bonded to the distal tip of the mandrel body tubing with a high temperature epoxy. The epoxy bond forms a taper extending from the outer diameter of the mandrel tubing down to the high resistance wire arc. The taper may assume the profile of tapered cone, or it may be a symmetrically tapered wedge. The taper facilitates insertion of the vascular sheath residing coaxially outside of the mandrel through the pericardium following its entry by the hot wire arc. When activated, the hot wire arc may reach a temperature of up to 2100° F. Due to its extremely limited mass, and the fluid environment on both sides of the pericardium, the hot wire arc cools immediately after passage through the pericardium, avoiding thermal injury to the heart inside the pericardium or other tissue outside of the pericardium, upon entry or exit through the pericardial sac.

In a third alternate embodiment, the heated mandrel may be an elongated stainless steel mandrel inserted into a standard electrosurgical pencil. The body of the mandrel is electrically insulated and contains an outer diameter dimensioned to be a slip fit with the inner lumen of a vascular sheath. The distal uninsulated tip of the mandrel is spherical or conical in shape, and it extends distal to the distal end of the vascular sheath. The electrosurgical pencil is powered by a radiofrequency generator, and a grounding pad attaches to the patient and connects to the generator.

The pericardial entry and exit system may also be useful for controlled entry and exit in additional anatomic locations that require passage through a fibrous membrane or layer. For example, in the percutaneous nephrolithotomy procedure, a sheath is placed into the patient's renal calyces via a small flank incision. A fibrous membrane called Gerota's fascia envelops the kidney, and passage through this membrane may be facilitated by use of a hot wire probe versus a standard sharp trocar or needle. Similarly, laparoscopic procedures require entry through abdominal fascial layers such as the linea alba in the midline, or the anterior and posterior rectus fascial layers lateral to the midline. A hot wire probe may allow controlled entry compared to the sharp tips of a pneumo-needle or a trocar obturator.

More specifically, the device is a catheter with a braided distal end that traverses the pericardium and deploys in an expanded fashion outside the pericardium. The anchor portion of the catheter is maintained in an expanded position by a proximal locking system whose outer diameter does not exceed the outer diameter of the main body of the catheter, enabling the anchor catheter to pass through a lumen in a balloon cannula implanted within the pericardial sac to fix the position of the balloon anterior to the ventricle of the heart. The proximal end of the anchor catheter is secured to the implantable reservoir at the proximal end of the balloon cannula, to prevent axial movement of the balloon cannula with respect to the anchor catheter.

Upon cyclical inflation and deflation of the balloon, it is observed that the balloon cannula migrates out of position over the left ventricle, and it skews towards the right side of the heart. This leads to a loss of left ventricular compression and ineffective left ventricular assistance. Therefore, it is desirable to provide an anchoring system for the tip of the balloon cannula. It is additionally desirable to provide an anchoring system for the ventricular assist balloon cannula that allows exchange of the balloon cannula while preserving the favorable position established by the original anchor system. The balloon has a finite life span; for example, one year. Provision of an anchor system that allows balloon cannula exchange simplifies and shortens subsequent balloon replacement procedures.

The proposed intrapericardial balloon cannula anchor system comprises a small diameter non-collapsible catheter body with a short distal section comprised of a braided sheath formed of multiple polymer strands. A rounded tip is attached to the distal end of the braided sheath, and a stainless-steel wire attached to the tip extends the length of the braided sheath and the catheter body. A length of stainless-steel tubing is bonded to the proximal portion of the catheter body, and the stainless-steel tubing extends approximately one centimeter proximal to the proximal end of the catheter. The steel wire inside the catheter is a slip fit with the inner diameter of the stainless-steel tube, and it protrudes several centimeters proximal to the proximal end of the stainless-steel tube. Traction on the stainless-steel wire while the catheter is held stationary causes the braided sheath to form an expanded disc. The braided sheath is maintained in its expanded configuration by crimping the stainless-steel tube onto the inner stainless-steel wire. Since the outer diameter of the stainless-steel tube approximates the inner diameter of the catheter, its profile following crimping is equal to the profile of the catheter body. The small uniform anchor catheter profile post deployment allows the ventricular assist balloon cannula to be advanced along the anchor catheter into proper position inside the pericardial sac.

The pericardial anchor contains a catheter body approximately 0.060″ in outer diameter, with a wall reinforced with a stainless-steel coil or braid to impart the high column strength needed to transform the distal braided section from a tubular structure to an expanded disc upon application of traction to the stainless-steel wire inside the catheter lumen. The braided sheath is formed of multiple strands of stiff polymeric or super-elastic metallic fibers braided in a crisscross fashion. The braid may be composed of 24 or 36 strands of polyethylene terephthalate (PET) or similar fibers approximately 0.010″ in outer diameter, or it may be formed of strands of Nitinol wire. The braided sheath has a length of approximately 15 mm. Its proximal portion may overlap the distal end of the catheter body by approximately 3 mm and be adhesive or heat bonded to the catheter. The distal portion of the braid overlaps the proximal portion of the rounded tip, and it may also be adhesive, or heat bonded to the polymeric or metallic rounded tip. A stainless-steel wire, approximately 0.025″-0.035″ in diameter is attached to the rounded tip and extends the length of the catheter and protrudes several centimeters proximal to the stainless-steel tube attached to the proximal end of the catheter. The distal end of the stainless-steel wire may be welded to the rounded tip, if the tip is metallic. If the tip is polymeric, the distal end of the stainless-steel wire may be flattened and a hole drilled in the flattened portion, or it may be looped and glued or cast into the rounded tip. The looped distal end of the stainless-steel wire may reside in the distal portion of the braided section and an ultraviolet curable adhesive may be injected and cured to attach the loop to the inside of the distal braid as well as to form the rounded tip.

The pericardial anchor is advanced into position through a vascular introducer sheath. Percutaneous subxiphoid entry into the pericardial sac is performed with a long hypodermic needle under fluoroscopic guidance, and a guidewire is inserted through the needle into the intrapericardial space. An introducer sheath and dilator are advanced over the guidewire into the pericardial sac, followed by removal of the guidewire. The sheath and dilator are advanced anterior to the surface of the heart into contact with the left lateral aspect of the pericardium at the inferior border of the left atrial appendage, axial force is applied to cause the sheath and dilator to puncture through the pericardium and exit into the left pleural cavity. A sharp stiff stylet may be inserted into the lumen of the dilator to increase its stiffness for pericardial exit. Entry by the sheath into the left pleural cavity is followed by removal of the inner dilator and advancement of the anchor catheter through the sheath, until the braided section of the anchor catheter extends out of the distal end of the sheath. The stainless-steel wire on the anchor catheter is pulled proximally to deploy the anchor disc, and the stainless-steel tube on the proximal end of the anchor catheter is crimped to lock the anchor disc in the expanded position. Excess length of stainless-steel wire protruding proximal to the stainless-steel tube is severed, and a short length of thin-walled polymer tubing that is an interference fit with the stainless-steel tube is advanced onto the crimped tube to cover any sharp edges that remain on the cut stainless-steel wire.

Following deployment of the anchor catheter, the ventricular assist balloon cannula is advanced along the shaft of the anchor catheter into position inside the pericardial sac. The balloon cannula contains an ancillary lumen that extends the length of the balloon and resides on the outer surface of the cannula body. This ancillary lumen accommodates the shaft of the pericardial anchor catheter. The balloon cannula is advanced until its distal tip contacts the inner pericardial surface at the exit site of the anchor catheter. The anchor catheter is pulled proximally to pinch the pericardium between the deployed anchor disc on the outside of the pericardium and the tip of the balloon cannula on the inside of the pericardium. The proximal end of the anchor catheter is inserted into a channel present in the side of the subcutaneous reservoir attached to the balloon cannula, and a setscrew extending into the lumen of the channel is tightened to fix the balloon cannula against axial movement with respect to the anchor catheter.

The expanding braided pericardial anchor catheter may also contain an exposed stainless-steel distal tip that may conduct radiofrequency energy to the pericardium at the point of contact, to allow exit through the pericardium with reduced axial force and increased control. In this version of a pericardial anchor catheter, the proximal end of the stainless-steel wire residing inside the catheter contains a 15 mm long section with an enlarged outer diameter that inserts into the distal receptacle of a standard electrosurgical pencil connected to a radiofrequency electrosurgical generator.

In a first specific embodiment, the present invention provides a pericardial access system comprising an introducer sheath, a mandrel, an electrode, and optionally a power supply. The introducer sheath has a proximal end, a distal end, and a lumen extending therethrough. The mandrel has a proximal end, a distal end, end a handle at the proximal end. The electrode is typically a wire electrode, such as a wire loop electrode, a U-shaped electrode, or a V-shaped electrode, and is disposed at the distal end of the mandrel. The power supply is configured to be detachably coupled to the electrode to apply a short burst of energy sufficient to allow the wire or other electrode to penetrate a pericardial sac of a patient.

In preferred examples, the power supply is located within the handle, typically comprising a rechargeable or replaceable battery. Alternatively, the power supply may located in a separate generator box and coupled to the handle by a cable.

In preferred examples, the mandrel is removably disposed in the lumen of the introducer sheath.

In a second specific embodiment, the present invention provides a method for accessing a pericardial space beneath a patient's pericardium. The method comprises penetrating a needle beneath the patient's xiphoid, advancing an introducer sheath over the needle to locate a distal end of the sheath proximate the pericardium, removing the needle from a lumen of the introducer sheath, advancing an electrode through the lumen, engaging the electrode against a target location on the pericardium, energizing the electrode to penetrate the pericardium and provide a hole therethrough, advancing the introducer sheath into the pericardial space beneath a patient's pericardium, removing the electrode from the lumen of the introducer sheath; and advancing an interventional tool through the lumen of the introducer sheath into the pericardial space.

In a third specific embodiment, the present invention provides an implantable cardiac assist catheter for use with an external drive unit. The implantable cardiac assist catheter comprises a catheter body having a proximal end, a distal end, and a lumen extending at least partly therethrough. A pneumatic effector, such as an inflatable balloon, bladder, or other structure, is disposed at the distal end of the catheter body and is configured to be implanted beneath a patient's pericardial sac (pericardium) and over a myocardial surface overlying the patient's left ventricle. An implantable port is attached or attachable at the proximal end of the catheter and is configured to receive a percutaneously introduced cannula. The port is connected to supply a driving gas received from the cannula through a gas lumen in the catheter body to drive the pneumatic effector, and an anchor catheter is configured to be percutaneously advanced into a patient's pericardial sac and comprises an anchor at its distal end which is configured to be expanded within the pericardial sac to stabilize the cardiac assist catheter during operation. Preferably, the catheter body of the implantable cardiac assist catheter is configured to be advanced over the anchor catheter via the lumen.

In preferred examples, the catheter body comprises a distal tip having a guidewire lumen with an entry port and an exit port, both ports being located distal to the pneumatic effector.

In preferred examples, the gas lumen is the only lumen in the catheter body between the proximal end and the pneumatic effector.

In preferred examples, the cardiac assist catheter further comprises an anchor disposed distally of the pneumatic effector on the catheter body.

In a fourth specific embodiment, the present invention provides a method for delivering and stabilizing a ventricular assist balloon cannula into a pericardial space beneath a patient's pericardium and over the patient's left ventricle. The method comprises advancing the ventricular assist balloon cannula beneath the patient's xiphoid to position a balloon over the left ventricle and advancing a pericardial anchor catheter through at least a portion of the ventricular assist balloon cannula so that a distal portion of the pericardial anchor catheter extends outwardly through a distal location on the pericardium. The distal portion of the pericardial anchor catheter is then anchored in a location external to the pericardium to thereby stabilize te position of the ventricular assist balloon cannula.

In preferred examples, the distal portion of the pericardial anchor catheter is anchored in a location external to the pericardium comprises expanding a disc-shaped anchor. For example, the distal portion of the pericardial anchor catheter may be anchored in a location external to the pericardium comprises penetrating an anchor into tissue or bone.

In preferred examples, the pericardial anchor catheter may be advanced through a tubular structure on a distal portion of the ventricular assist balloon cannula. Often, the tubular structure is disposed beneath the balloon of the ventricular assist balloon cannula.

### BRIEF DESCRIPTION OF THE DRAWINGS

FIG. 1 illustrates conventional needle entry through the pericardial sac via a subxiphoid approach.

FIG. 2 depicts guidewire insertion following successful needle insertion into the pericardial space.

FIG. 3 depicts vascular sheath and dilator insertion over the guidewire into the pericardial space and dilator and sheath exit from the left lateral border of the pericardial sac.

FIG. 4 shows anchor catheter deployment through the vascular sheath.

FIGS. 5A to 5C show the individual and assembled components of the pericardial entry and exit system.

FIGS. 6A to 6D illustrate the construction of the hot wire inner stylet with its control handle.

FIG. 7 illustrates the components encountered in a conventional electrosurgical pencil system.

FIGS. 8A to 8C depict the components added to a standard electrosurgical pencil system for application to the pericardial entry and exit technique.

FIG. 9 shows the configuration and components included in the pericardial anchor catheter.

FIGS. 10A to 10D depict the steps in deployment and fixation of the expanding disc of the pericardial anchor catheter.

FIGS. 11A (top view) and 11B (side view) show the configuration of the ventricular assist balloon cannula with its attached subcutaneous reservoir.

FIGS. 12A (top view) and 12B (side view) illustrate subxiphoid pericardial entry with a needle and vascular sheath and dilator insertion into the pericardial sac.

FIGS. 13 to 17 show deployment of vascular sheath and dilator exiting the left lateral border of the pericardium, and advancement of the anchor catheter through the sheath into the left pleural cavity and expanding disc in the left pleural cavity and attachment of the proximal end of the anchor catheter to the subcutaneous reservoir.

### DETAILED DESCRIPTION OF THE DRAWINGS

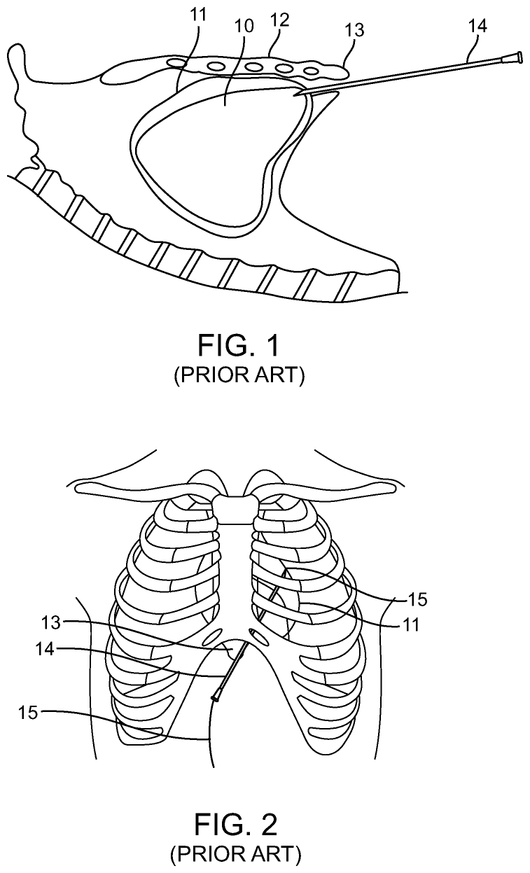

FIG. 1 is a sagittal section through a patient's torso, depicting the conventional technique of entry through the pericardial sac 11 via insertion of needle 14 inferior to the xiphoid process 13 of sternum 12. The pericardium 11 may be in apposition with the apex of the heart 10, and upon advancement of needle 14 to enter the pericardium 11, the sharp tip of needle 14 may easily puncture or lacerate the heart 10.

FIG. 2 is a diagram depicting an anterior view of a patient's chest during conventional pericardial entry, illustrating insertion of a guidewire 15 into the pericardium (pericardial sac) 11 following entry by needle 14 advanced inferior to the xiphoid process 13. Guidewire 15 is inserted through needle 14 under fluoroscopic guidance.

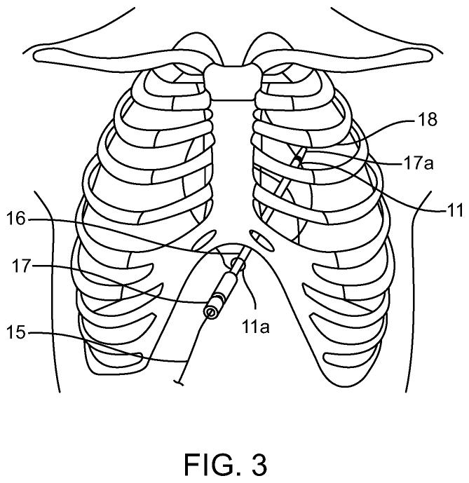

FIG. 3 illustrates the technique of pericardial exit, using conventional cardiovascular interventional instrumentation. With the guidewire 15 positioned inside the pericardial sac 11, the hypodermic needle is removed, and a vascular sheath 16 with its intraluminal tapered dilator 17 inserted through a penetration 11a in the pericardium 11 over the guidewire 15. With the guidewire 15 retracted inside the dilator 17, a dilator tip 17a is used to exit the left lateral border of the pericardium 11. Significant force must be applied to cause the tip of the dilator 17 to puncture through the pericardial membrane 11, and the dilator tip 17 may easily enter the lung 18 after exiting the pericardial sac 11.

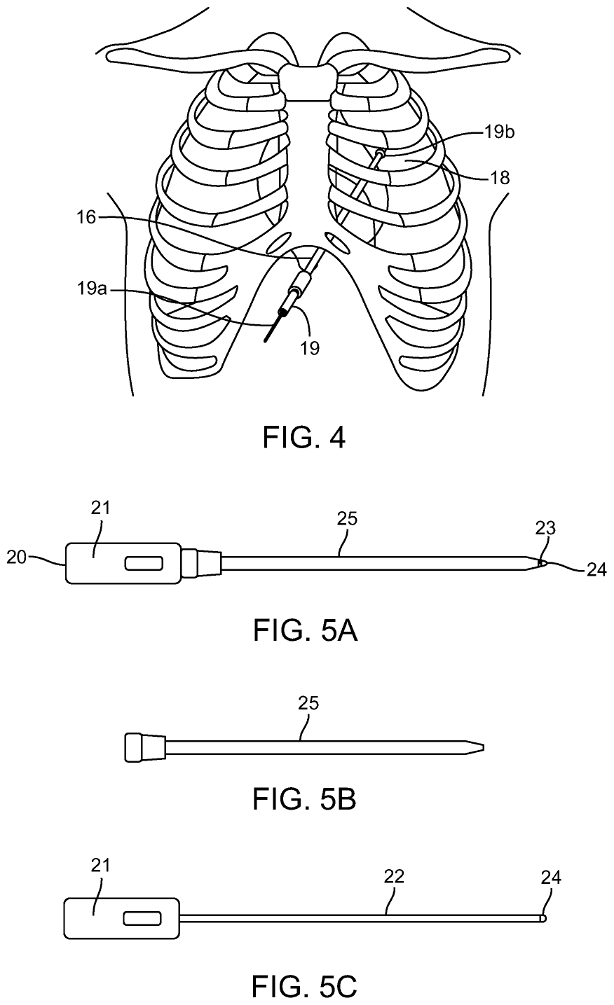

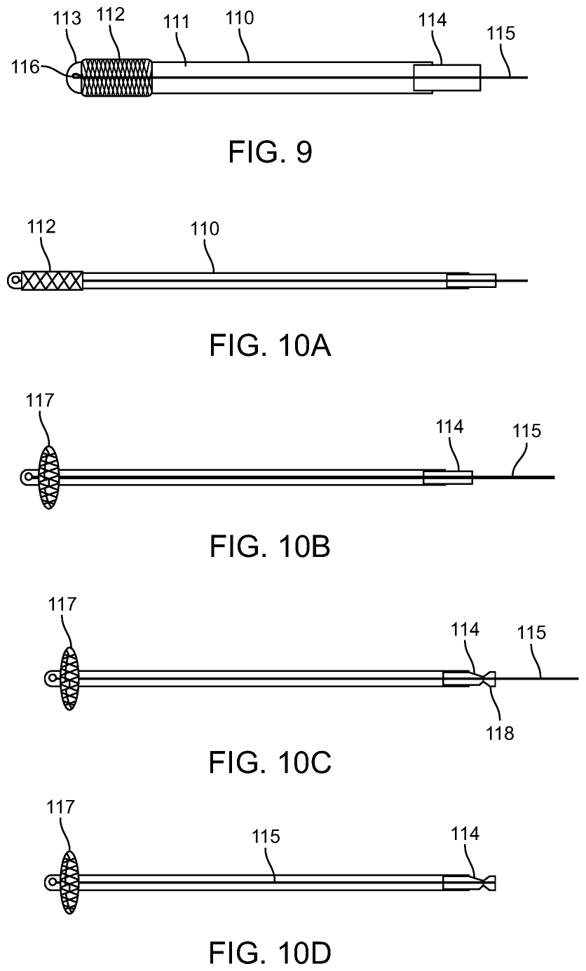

FIG. 4 illustrates the deployment of a pericardial anchor 19 through the vascular sheath 16 following removal of the guidewire 15 and the dilator 17. The tip 91b of pericardial anchor 19 is expanded in the pleural cavity adjacent the lung 18, typically by retracting a wire 19b that foreshortens and expands the tip 19b as described in more detail below. Following deployment of the pericardial anchor tip 19b, the vascular sheath 16 is removed from the body. The steps shown in FIGS. 3 and 4 are described further with respect to FIGS. 9 to 17 below.

FIG. 5A illustrates a pericardial entry and exit system 20 in accordance with the present invention, shown in its assembled configuration. A control handle 21 is attached to the proximal end of a mandrel 22 that extends the length of the vascular introducer sheath 25. The mandrel 22 contains a distal tapered portion that protrudes out of the distal end of the introducer sheath 25. A rounded wire arc 24 is present on the distal end of the mandrel 22. The wire arc 24 is constructed of a high electrical resistance wire such as Nichrome, a nickel chromium alloy. FIG. 5B illustrates the introducer sheath 25 that resides outside the mandrel 22 component of the device. Introducer sheath may contain a step 26 of increased diameter that resides approximately 7-10 mm proximal to the distal end of the sheath 25. Step 26 may possess a height that is 1-2 mm greater than the distal surface of the introducer sheath 25, and it serves as a stop against the pericardium during device insertion, to avoid excessive advancement of the sheath 25 into the lung tissue. FIG. 5c shows the mandrel 22 that contains an outer diameter that is a slip fit with the inner diameter of the introducer sheath 24. The mandrel 22 is partially rigid, and it supports the advancement of introducer sheath 25 through tissue and pericardial passage. The handle 21 attached to the proximal end of mandrel 22 is grasped by the physician, and a switch controls heating of the wire arc 24.

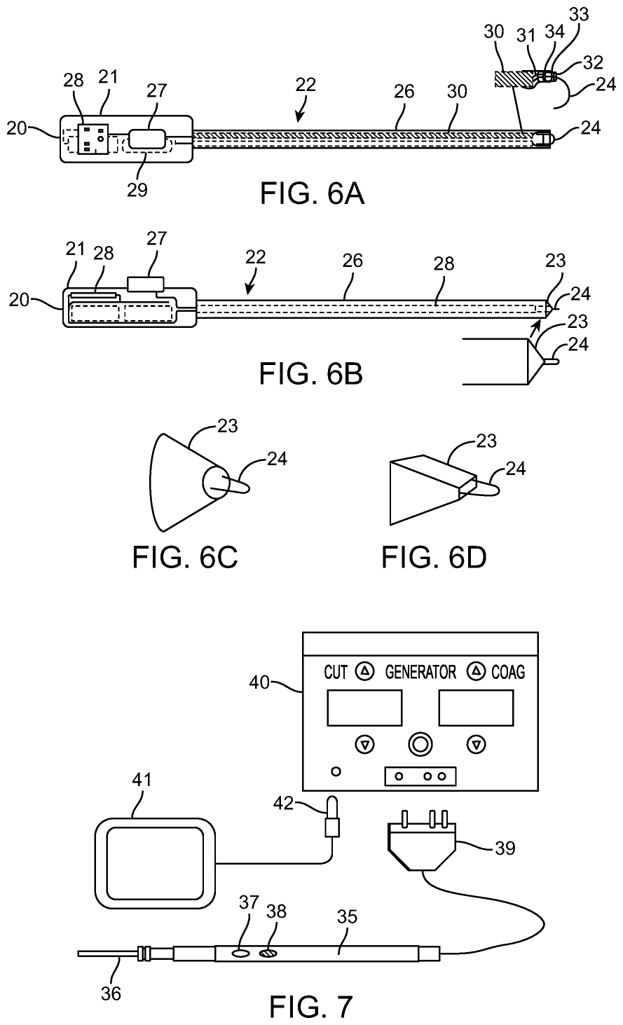

FIG. 6a shows a top view of the pericardial entry and exit system 20. An elongated mandrel 27 extends out of the handle 21 that contains a push button power switch 28 and batteries 30 to power the device. A timer circuit 29 may also be incorporated in the circuit in handle 21. If present, the timer circuit 29 turns off power 1 to 2 seconds after the power switch 28 is depressed, regardless of whether the switch 28 continues to be depressed. If timer circuit 29 is not present, the surgeon manually releases power switch 28 when a sudden release of resistance is observed as the hot wire arc 24 enters or exits the pericardium. The mandrel 27 is a semi-flexible tubular structure approximately 3 mm in outer diameter. Its lumen contains two electrically insulated wire electrodes 31 that extend to the distal tip of mandrel 27. The enlarged view of the distal end of the mandrel 27 shows that the bare uninsulated distal wire end 32 of electrode 31 enters the proximal end of metal tube 33, while the proximal end of high resistance wire arc 24 enters the distal end of metal tube 33. Crimps 34 in metal tube 33 fixate wire end 32 and wire arc 24 in conductive continuity. A polymer heat shrink tube 35 covers and electrically insulates the crimped metal tube 33. A similar connection is formed in the second wire electrode 31 inside mandrel 27. An adhesive is applied to the distal end of mandrel 27 to fixate the assembly such that wire arc 24 protrudes distal to the distal end of mandrel 27. FIG. 6b is a side view of pericardial entry and exit system 20. The distal end of mandrel 27 contains a tip 23 that tapers from the outer diameter of mandrel 27 down to the diameter of the wire in wire arc 24. Tapered tip 23 facilitates advancement of mandrel 27 through the pericardium following initial entry by wire arc 24. The tapered tip 23 may be formed by the polymeric adhesive material used to fixate the crimped tube 33 and wire arc 24 assembly to the distal end of mandrel 27, or it may be a molded polymeric part. FIG. 6c shows that the tapered tip 23 may be a frustoconical shape. FIG. 6d shows that alternatively, the tapered tip 23 may a truncated pyramidal shape.

FIG. 7 depicts a electrosurgical system, composed of an electrosurgical pencil 36 containing a detachable distal stainless steel blade 37, a button switch 38 that delivers a radiofrequency waveform for cutting tissue, a button switch 39 that delivers a radiofrequency waveform to coagulate bleeding tissue, and a connector 40 that connects the electrosurgical pencil 36 to the radiofrequency generator unit 41. A grounding pad 42 that is attached to the surgical patient is part of the system, and it contains a proximal connector 43 that connects the grounding pad 42 to the electrosurgical generator 41. The radiofrequency embodiment of the pericardial entry and exit system may replace the stainless-steel blade 37 with an elongated insert that fits the inner lumen of a vascular sheath.

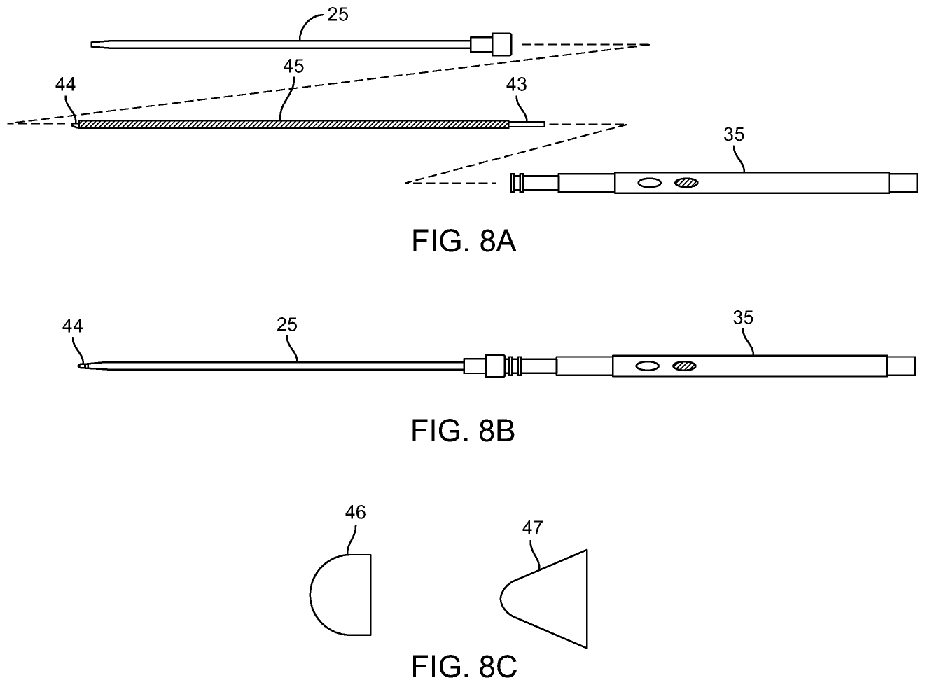

FIG. 8A shows an elongated insert 44 dimensioned to fit into the distal receptacle of a standard electrosurgical pencil 36 and to act as a mandrel inside a vascular sheath 25. Insert 44 consists of a stainless-steel inner core that is 3/32″ in diameter. Its proximal 2 cm long portion and its distal tip 45 are uninsulated, while the remainder of its length is covered with polymeric electrical insulation 46. FIG. 8B shows the assembled components of the radiofrequency pericardial entry and exit system consisting of the vascular sheath 25 positioned coaxially on the elongated insert 43 that is placed into the distal portion of electrosurgical pencil 36. FIG. 8C is an enlarged view of the tissue contacting distal portion of tip 45, showing that it may be hemispherical 47, or it may assume a tapered conical configuration 48.

FIG. 9 illustrates the components that form a pericardial anchor catheter 110. The distal end of a reinforced catheter body 111 is attached to the proximal end of a braided sheath 112. A rounded cap 113 is attached to the distal end of braided sheath 112. A stainless-steel tube 114 is attached to the inner lumen of the proximal end of catheter body 111. A stainless-steel wire 115 extends the length of the braided sheath 112, catheter body 111 and stainless-steel tube 114, and further extends proximally out of the proximal end of stainless-steel tube 114 by a distance of 1 to 2 cm.

The distal end of stainless-steel wire 115 may be formed into a wire loop 116, for example by bending the wire 115 or by flattening the distal end of wire 115 and drilling a hole in the flattened portion. Wire loop 116 may be permanently attached to rounded cap 113 by means of an adhesive if the rounded cap 113 is formed of polymeric material or via a weld joint if the rounded cap 113 is metallic. Alternatively, the rounded cap 113 may be formed by placing and curing a drop of ultraviolet-cure adhesive on wire loop 116 and the distal end of braided sheath 112, providing a rigid rounded cap 113.

FIG. 10A shows the pericardial anchor catheter 110 in its resting configuration with the braided sheath 112 in a relaxed state. By proximally retracting a stainless-steel wire 115 or other tether relative to an outer, stationary stainless-steel tube 114, the relaxed braided sheath 112 is axially foreshortened and radially expanded to become a disc-shaped anchor sheath 117, as shown in FIG. 10B. The anchor sheath 117 may maintained in its disc configuration by locking the stainless-steel wire 115 relative to the stationary stainless-steel tube 114, for example by applying a crimp 118 to the stainless-steel tube 114, as shown in FIG. 10C. The excess length of stainless-steel wire 115 may be severed flush with the proximal end of stainless-steel tube 114 to complete deployment of pericardial anchor catheter 110, as shown in FIG. 10D.

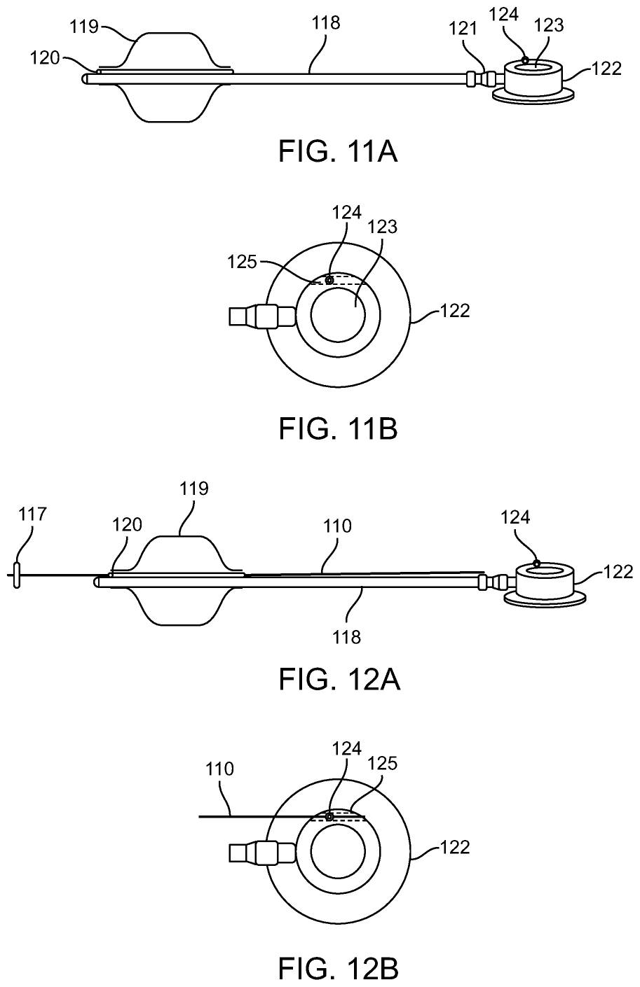

FIG. 11A depicts an implantable ventricular assist balloon cannula 118 having a distal ventricular assist balloon 119 and a short distal tube 120 extending beneath the balloon 119 and over an external distal surface of the cannula 118. The short distal tube 120 has a lumen to accommodate the pericardial anchor catheter 117 to maintain positioning of the balloon cannula 18. Ventricular assist balloon cannula 118 contains a proximal fitting 121 that connects to an implantable reservoir 122, forming an airtight assembly between an internal chamber of the implantable reservoir 122, the cannula 118 and balloon 119. A superior face 123 of implantable reservoir 122 comprises an elastomeric membrane that may be penetrated by a large bore hypodermic needle inserted through the patient's skin to deliver air to the ventricular assist balloon cannula 118 supplied by a pump unit external to the patient.

FIG. 11B is an enlarged top view of the implantable reservoir 122, showing a channel 125 extending through a side wall of a housing of the reservoir and a setscrew 124 extends from the top of the reservoir housing into the lumen of the channel 25. The lumen of channel 125 will accommodate the pericardial anchor catheter 117 and setscrew 124 will allow fixation of the pericardial anchor catheter 117 with respect to implantable reservoir 22.

FIG. 12A shows a full assembly of the pericardial anchor catheter 110 residing inside the lumen of the short distal tube 120 of implantable balloon cannula 18, following expansion of pericardial anchor disc 17. FIG. 12B is an enlarged top view of implantable reservoir 122, depicting the proximal end of pericardial anchor catheter 10 residing in channel 25 in the side wall of implantable reservoir 22, fixed in position by setscrew 24.

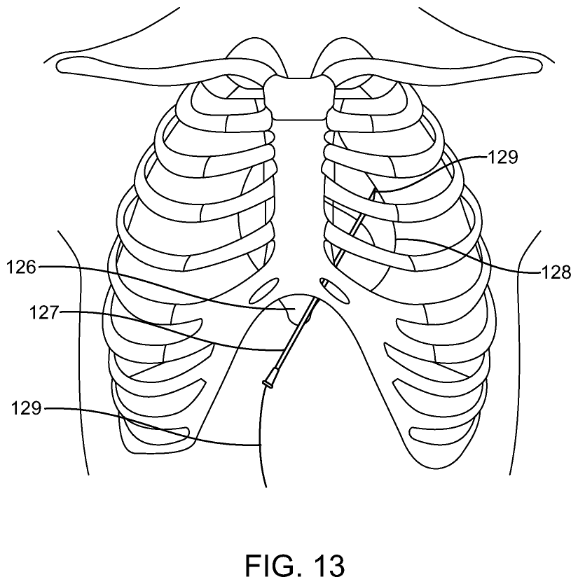

FIGS. 13 to 17 show placement of a ventricular assist balloon cannula and pericardial anchor system via percutaneous pericardial entry in accordance with the principles of the present invention. In FIG. 13, a hypodermic needle 127 is inserted inferior to the xiphoid process 126 of the patient's sternum and is advanced under fluoroscopic guidance to enter the pericardial sac (pericardium) 128 into the pericardial cavity, followed by advancement of a guidewire 29 into the pericardial cavity (intrapericardial space).

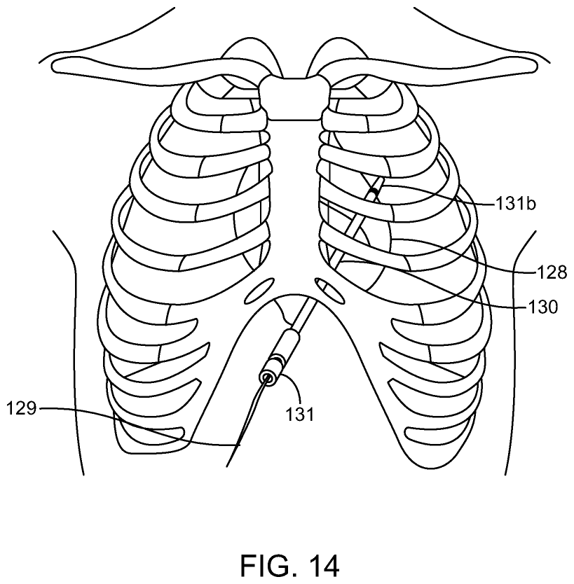

As shown in FIG. 14, a vascular sheath 130 and its inner dilator 131 are introduced over the guidewire 129 into the intrapericardial space beneath the pericardial sac 128 with an internal dilator (having a dilator tip 131a) and a distal portion of the sheath 130 exiting through the left border of the pericardium 128. Guidewire 129 is then withdrawn into the lumen of the inner dilator.

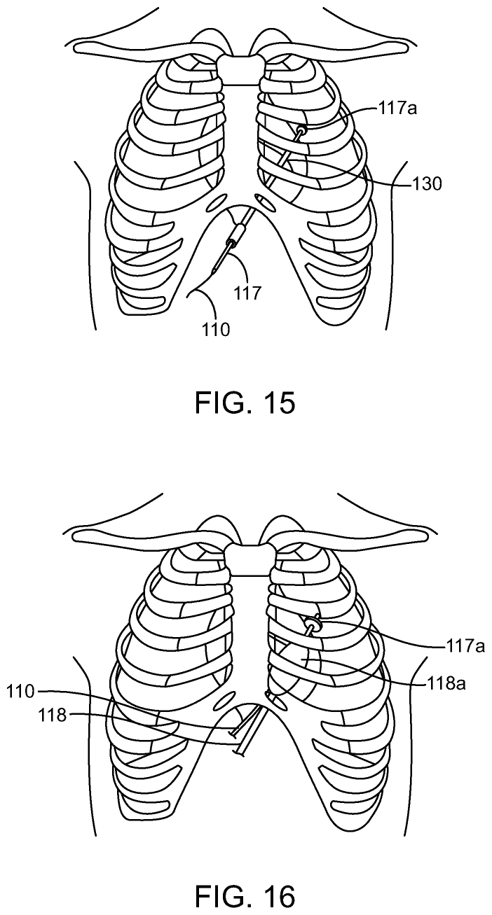

As shown in FIG. 15, following removal of the inner dilator 131, the pericardial anchor catheter 110 is then introduced through the vascular sheath 130, and the pericardial anchor disc 117a is expanded and deployed outside of the pericardium 28.

FIG. 16 depicts advancement of the ventricular assist balloon cannula 118 along the pericardial anchor catheter 110 to position inflatable balloon 118a over the left ventricle and beneath the patent's ribs.

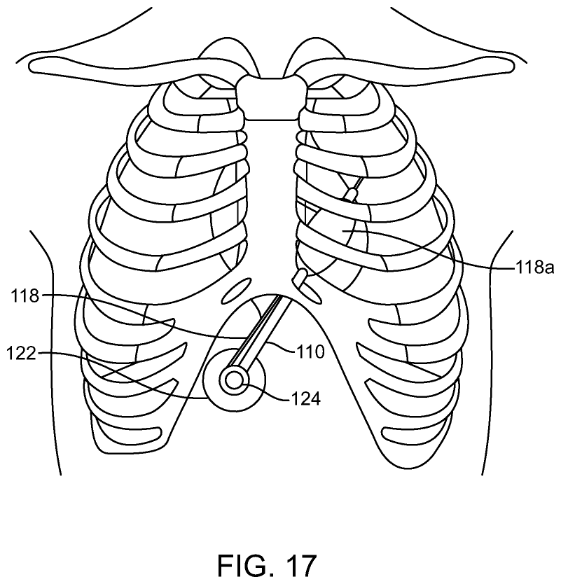

FIG. 17 depicts subcutaneous reservoir 122 attached to ventricular assist balloon cannula 118 and the proximal portion of pericardial anchor 110 locked into subcutaneous reservoir 122 via setscrew 124.

Although certain embodiments or examples of the disclosure have been described in detail, variations and modifications will be apparent to those skilled in the art, including embodiments or examples that may not provide all the features and benefits described herein. It will be understood by those skilled in the art that the present disclosure extends beyond the specifically disclosed embodiments or examples to other alternative or additional examples or embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments and examples may be made and still fall within the scope of the present disclosure. Accordingly, it should be understood that various features and aspects of the disclosed embodiments can be combined with or substituted for one another in order to form varying modes or examples of the present disclosure. Thus, it is intended that the scope of the present disclosure herein disclosed should not be limited by the particular disclosed embodiments or examples described above. For all of the embodiments and examples described above, the steps of any methods for example need not be performed sequentially.

1.-8. (canceled)

9. A method for accessing a pericardial space beneath a patient's pericardium, said method comprising:
inserting a needle beneath the patient's xiphoid process;
penetrating the pericardial space with the needle;
advancing a guidewire inside the needle into the pericardial space;
removing the needle from beneath the patient's xiphoid process;
advancing a catheter into the pericardial space beneath the patient's pericardium;
and
advancing an interventional tool through the lumen of the catheter into the pericardial space.

10. An implantable cardiac assist catheter for use with an external drive unit, said implantable cardiac assist catheter comprising:
a catheter body having a proximal end, a distal end, and a lumen extending at least partly therethrough;
a pneumatic effector at the distal end of the catheter body and configured to be implanted beneath a patient's pericardial sac and over a myocardial surface overlying the patient's left ventricle;
an implantable port at the proximal end of the catheter and configured to receive a percutaneously introduced cannula, said port being connected to supply a driving gas received from the cannula through a gas lumen in the catheter body to the pneumatic effector; and
an anchor catheter configured to be percutaneously advanced into a patient's pericardial sac and having an anchor at its distal end and configured to be expanded within the pericardial sac;
wherein the catheter body of the implantable cardiac assist catheter is configured to be advanced over the anchor catheter via the ancillary lumen.

11. The implantable cardiac assist catheter of claim 10, wherein the catheter body comprises a distal tip having a guidewire lumen with an entry port and an exit port, both ports being located distal to the pneumatic effector.

12. The implantable cardiac assist catheter of claim 10, wherein the gas lumen is the only lumen in the catheter body between the proximal end and the pneumatic effector.

13. The implantable cardiac assist catheter of claim 10, further comprises an anchor disposed distally of the pneumatic effector on the catheter body.

14. A method for delivering and stabilizing a ventricular assist balloon cannula into a pericardial space beneath a patient's pericardium and over the patient's left ventricle, said method comprising:
advancing a pericardial anchor wire into the pericardial space, wherein a distal portion of the pericardial anchor wire extends outwardly through a distal location on the pericardium;
anchoring the distal portion of the pericardial anchor wire in a location external to the pericardium to thereby stabilize the position of the ventricular assist balloon cannula; and
advancing the ventricular assist balloon cannula beneath the patient's xiphoid process along the pericardial anchor wire to position a balloon over the left ventricle.

15. The method of claim 14, wherein anchoring the distal portion of the pericardial anchor catheter in a location external to the pericardium comprises expanding a disc-shaped anchor.

16. The method of claim 14, wherein anchoring the distal portion of the pericardial anchor catheter in a location external to the pericardium comprises penetrating an anchor into tissue or bone.

17. The method of claim 14, wherein the pericardial anchor catheter is advanced through a tubular structure on a distal portion of the ventricular assist balloon cannula.

18. The method of claim 17, wherein the tubular structure is disposed beneath the balloon of the ventricular assist balloon cannula.

19. The method of claim 9, wherein the interventional tool comprises a fluid tight reservoir.

20. The method of claim 19, wherein the fluid tight reservoir comprises an expandable balloon.

21. The method of claim 19, wherein the fluid tight reservoir is configured to be inflated via an air pump using a needle penetrated through the patient's skin and into the fluid tight reservoir.

22. The method of claim 9, wherein the interventional tool comprises a ventricular assist balloon cannula.
